(19) United States
(12) Patent Application Publication
(10) Pub. No.: US 2021/0341417 A1
(43) Pub. Date: Nov. 4, 2021

(54) CALIBRATION DEVICE, FLEXIBLE BAG CONTAINING COMPONENTS OF A CALIBRATION DEVICE, AND METHOD FOR CALIBRATING A SENSOR

(71) Applicant: Endress+Hauser Conducta GmbH+Co. KG, Gerlingen (DE)

(72) Inventor: Andreas Löbbert, Waldheim (DE)

(21) Appl. No.: 17/238,916

(22) Filed: Apr. 23, 2021

(30) Foreign Application Priority Data
Apr. 30, 2020 (DE) 10 2020 111 802.3

(51) Int. Cl. G01N 27/416; G01N 27/404; G01N 27/413

(52) U.S. Cl. CPC G01N 27/4163; G01N 33/0006; G01N 27/413; G01N 27/404

(57) ABSTRACT

Disclosed is a calibration device for calibrating a sensor designed to determine a proportion of a gas in a gas mixture. The device includes a container having an electrolyte liquid and two electrodes immersed in the electrolyte liquid; a gas-generation chamber arranged in the container such that the gas-generation chamber partially surrounds a first electrode of the two electrodes and electrolyte liquid is in the gas-generation chamber; and a calibration chamber with a guide into which guide the sensor can be introduced, wherein the calibration chamber is connected via at least one closable opening to the gas-generation chamber, wherein an electric voltage is applied to the electrodes and by electrolysis separates a gas mixture containing the gas to be detected. The gas mixture flows from the gas-generation chamber through the open closable opening into the calibration chamber. Also disclosed is a flexible bag for providing components of the calibration device.

### CROSS-REFERENCE TO RELATED APPLICATION

The present application is related to and claims the priority benefit of German Patent Application No. 102020111802.3, filed on Apr. 30, 2020, the entire contents of which are incorporated herein by reference.

### TECHNICAL FIELD

The present disclosure relates to a calibration device for calibrating a sensor, said sensor being designed to determine a proportion of a gas to be detected in a gas mixture. The present disclosure furthermore relates to a flexible bag for providing components of a calibration device and to a method for calibrating a sensor.

### BACKGROUND

Electrochemical or optochemical sensors are used in laboratory and process measurement technology for the analysis of measured media in many fields of chemistry, biochemistry, pharmacy, biotechnology, food technology, water management, and environmental metrology. An electrochemical sensor is, for example, a potentiometric (for example, an ion-selective electrode (ISE), such as the known pH glass electrode) or an amperometric sensor (for example, an amperometric disinfection sensor). Further examples are those based on electrolyte-insulator-semiconductor layered stacks (EIS for short), such as ISFET sensors, inductively or capacitively operating conductivity sensors or (spectro-)photometrically operating sensors, such as turbidity sensors or gas sensors. Gas sensors often take the form of optical sensors; see, for example, DE 10 2014 112 972 A1 or DE 10 2019 120 658 A1. Gas sensors by means of which traces of a gas, for example, oxygen, can be detected are also referred to as trace sensors.

Such sensors must be calibrated, verified, and/or adjusted, for example, upon being put into operation or from time to time. Here, for example, a reference sensor serving as a reference or a measured value determined therewith and assumed to be correct is used as reference value. As an alternative or in addition to the use of a reference sensor, so-called calibration standards are also used. By means of a calibration standard, at least one process variable determinable with the sensor, especially, an analysis measurand, is provided in a controlled manner as a reference for the sensor and possibly also for the reference sensor, whereby the sensor can be calibrated, verified, and/or adjusted.

Calibration usually refers to the detection of a deviation between the measured value measured with the sensor and the reference value assumed to be correct. Verifying also includes the determination of the deviation and the evaluation thereof. Adjustment means adapting the sensor in such a way that its measured value matches the reference value. Calibrating, verifying, and/or adjusting generally takes place at least when the sensor is put into operation or, if necessary, even repeatedly, for example, at regular calibration intervals, if, for example, an aging-related drift of the sensor is to be assumed.

In practice, a plurality of different reference values is often used in calibrating, verifying, and/or adjusting a sensor (also: multi-point calibration). In a multi-point calibration, a plurality of calibration standards is used which have different, especially, predetermined, values for a process variable, especially, an analysis measurand, that can be determined the sensor (or the reference sensor). The sensor to be calibrated, verified, and/or adjusted and is then brought into contact with the calibration standard for the case of using a reference sensor, for example, consecutively or simultaneously with the reference sensor.

For gas sensors, calibration standards are available as test gas mixtures. These generally require dealing on site with gas cylinders for the production and/or provision of test gases. This is not always practical or is even ruled out, for example, in potentially explosive areas in which the use of inflammatory gases is in principle undesirable. This is especially true when a calibration near the process or on site (also: field calibration) is desired or required. Since the transportation of gas cylinders by air freight is moreover not permitted, a timely provision of test gas mixtures is not always possible.

### SUMMARY

The object of the present disclosure is therefore to provide a calibration device and a method for calibrating a gas sensor, by means of which a calibration standard can be provided in a simple and reliable manner.

With regard to the calibration device, the object is achieved by a calibration device for calibrating a sensor, said sensor being designed for determining a proportion of a gas to be detected in a gas mixture, comprising:
a container having an electrolyte liquid and two electrodes immersed in the electrolyte liquid;
a gas-generation chamber arranged in the container such that the gas-generation chamber at least partially surrounds a first electrode of the two electrodes and electrolyte liquid is arranged in the gas-generation chamber,
a calibration chamber with a guide, into which guide the sensor to be calibrated can be at least partially introduced,

wherein the calibration chamber is connected via at least one closable opening to the gas-generation chamber,

wherein by application of an electric voltage to the electrodes, the electrolyte liquid at the first electrode by means of electrolysis separates a gas mixture containing the gas to be detected,

which gas mixture flows from the gas-generation chamber through the open closable opening into the calibration chamber.

The sensor is, for example, an electrochemical sensor and/or an optochemical gas sensor, especially, an oxygen sensor.

Advantageously, a calibration standard for the gas sensor is produced by means of the gas mixture produced at the first electrode. Here, for example, the calibration chamber is arranged directly above an upper end of the gas-generation chamber so that the gas mixture rises from the gas-generation chamber into the calibration chamber.

In this case, the closable opening between the calibration chamber and the gas-generation chamber can, especially, even be reclosable, i.e., it can be closed/opened several times.

The advantages of the present disclosure are as follows:
The test gases produced according to the present disclosure and serving as calibration standards can be generated very easily by chemical decomposition from the electrolyte during electrolysis;
The electrolytic production of test gases, especially, for an oxygen trace sensor, requires comparatively low voltages so that, for example, a commercially available battery can be used as a power supply or source;
With the calibration device according to the present disclosure, test gases with a predeterminable proportion of the gas to be detected can be adjusted. The proportion can be adjusted via the electrolysis parameters (e.g., the electrolyte liquid used, the voltage applied at the electrodes, the electrode material, etc.). Preferably, a test gas with a gas concentration which lies in the middle of the measuring range of the gas sensor can thus be produced, for example. In contrast, in the prior art, when oxygen is supplied via gas cylinders, only calibration standards with proportions of, for example, 100% or 0% oxygen are used, in the latter case the oxygen being eliminated from the system by chemical reaction. The present disclosure thus enables an improved calibration since a preferred, predetermined gas concentration can be adjusted.

At least the sensor to be calibrated can be at least partially introduced into the guide, i.e., at least with a section having a sensitive component of the sensor, so that a calibration is possible.

If a reference sensor is used, the latter can optionally be introduced into the same guide, for example, at the same time or afterwards. Optionally, the calibration chamber can also have an additional guide provided for the reference sensor.

In one embodiment of the present disclosure, the electrolyte liquid separates at the first electrode a gas mixture containing oxygen (O2) as the gas to be detected, the gas mixture especially having an oxygen proportion of 0.001 to 21 percent by volume. In this case, the gas sensor is therefore an oxygen sensor.

In one embodiment of the present disclosure, the electrolyte liquid is an alkaline urea solution and the first electrode is the anode, wherein the applied electric voltage has a value from a range of 0.2 to 1.9 volts, and wherein the oxygen proportion is adjustable via the applied electric voltage.

The oxygen O2 as the gas 2 to be detected is here generated at the first electrode from hydroxygen or oxyhydroxides, which (oxy)hydroxides are in turn separated, for example, from a catalyst added to the electrolyte liquid and/or from a hydroxide of the first electrode.

This is especially an alkaline urea solution with a noble gas, for example, an argon-purged alkaline urea solution.

Voltages of 0.2 to 1.9 volts can be provided using commercially available batteries.

In one embodiment of the present disclosure, the calibration device has a voltage converter for reducing the applied voltage, especially, to a value in a range between 0.2 and 1.4 volts. Overvoltages and the formation of by-products, such as other gases in the gas mixture, are reduced by the voltage converter. Especially, the range 0.5 to 1.4 volts is preferred.

In one embodiment of the present disclosure, the at least one closable opening has a liquid-impermeable and gas-permeable membrane so that, when the opening is open, the gas mixture flows from the gas-generation chamber into the calibration chamber via the liquid-impermeable and gas-permeable membrane and the electrolyte liquid remains in the gas-generation chamber.

Suitable materials for the membrane are, for example, PTFE, PVDF, PVC, cellulose acetates. Furthermore, even porous membranes are conceivable, for example, with a pore size of more than 0.2 μm (micron). The membrane allows especially the gas mixture or at least the gas to be detected to pass through in a substantially unhindered manner. In this case, the membrane especially has a water intrusion pressure of at least 1 bar so that it has sufficient permeability for the gas mixture or at least for the gas to be detected with a simultaneous barrier effect for the electrolyte liquid.

In one embodiment of the present disclosure, the at least one closable opening has a valve. By means of a valve (in addition to the membrane or as an alternative to the membrane), the flow of the gas mixture from the gas-generation chamber into the calibration chamber can be controlled or regulated particularly easily.

In one embodiment of the present disclosure, the gas-generation chamber is designed as a first hollow body and the calibration chamber as a second hollow body.

Especially in each case, it is a cylindrical hollow body, wherein the first hollow body is designed, for example, as a downwardly open tube, which is arranged around the first electrode. As a result, the electrolyte liquid can always flow in and the gas mixture generated is held in the vicinity of the first electrode so that it can flow from the gas-generation chamber into the calibration chamber.

Suitable material for the first hollow body is, for example, a plastic or glass, stainless steel, etc.

Suitable also as material for the second hollow body is, for example, a plastic, a plastic or glass, stainless steel, etc.

In one embodiment of the present disclosure, a wall of the first hollow body and a wall of the second hollow body each have at least one recess, wherein the at least one closable opening between the gas-generation chamber and the calibration chamber is formed in that the recess(es) in the wall of the first hollow body can be brought into congruence with the recess(es) in the wall of the second hollow body by means of a relative movement of the two hollow bodies.

The relative movement involves, for example, a rotation and/or a displacement along a common longitudinal axis of the two hollow bodies. By performing the relative movement, the opening can thus be opened and closed again, wherein the two positions “open/closed opening” can be recognized by a mechanical latching or by a display for the user.

In one embodiment of the present disclosure, the calibration chamber has a volume of less than 50 ml, especially, less than 20 ml and preferably a volume of less than 10 ml. In such a small miniature calibration chamber, there is essentially no dead space so that an optimum flow of the gas mixture, and thus also of the test gas, onto the sensor to be calibrated can be assumed.

In one embodiment of the present disclosure, a catalyst is added to the electrolyte liquid, especially, a catalyst having metallic salts of the transition metals of the fourth period. The catalyst used is thus, for example, a salt of nickel (i.e., nickel hydroxide), cobalt, or iron, etc.

In one embodiment of the present disclosure, at least one of the electrodes has a metal, the metal being selected from the group of the following or combinations thereof: platinum, titanium, iridium, nickel, ruthenium.

The metal can be present, for example, as an inorganic or organic metal compound, for example, as a salt of one of the metals mentioned above.

In one embodiment of the present disclosure, the calibration chamber has a closure by means of which the calibration chamber can be sealed off in a substantially gas-tight manner from the environment, and/or the at least one closable opening is sealed in a substantially gas-tight manner when the opening is closed.

In one embodiment of the present disclosure, the at least one closable opening has a metal-coated film, especially, a metal-coated polymer film, for sealing off the calibration chamber from the gas-generation chamber in a substantially gas-tight manner, and/or the closure has a metal-coated film, especially, a metal-coated polymer film, for sealing off the calibration chamber from the environment in a substantially gas-tight manner.

The metal-coated film as a flexible connecting element between the gas-generation chamber and the calibration chamber serves for sealing the opening during opening or closing or for sealing the closure. Metal-coated films have excellent gas impermeability and are therefore suitable for sealing.

In one embodiment of the present disclosure, the closure and the at least one closable opening are mechanically coupled to each other via a closure mechanism in such a way that via a single operation of the closure mechanism, the closure and the at least one closable opening can be operated substantially simultaneously.

In a development of this embodiment, a switch of a power supply of the electrodes is mechanically coupled to the closure mechanism such that the power supply of the electrodes can be operated via the single operation of the closure mechanism.

For the opening and the closure, “can be operated” means, especially, “can be closed or opened” and for the power supply, it means “can be switched off or adjusted.”

In one embodiment of the present disclosure, the calibration device has a pressure compensation element which is designed to compensate for a pressure rise in the calibration chamber.

The pressure compensation element compensates for a critical pressure increase caused by the inflow of the gas mixture into the calibration chamber. A suitable pressure compensation element is, for example, an elastomer which is arranged in the calibration chamber. The pressure compensation element can also be arranged in a separate pressure compensation chamber which communicates with the calibration chamber. In this case, if necessary, the pressure compensation chamber can communicate with the calibration chamber only in the event of a critical overpressure, in that, for example, the calibration chamber is connected to the pressure compensation chamber by an overpressure valve which opens starting from a predetermined overpressure (e.g., 2 bar) and only then lets the gas mixture pass through.

Alternatively or in addition to the pressure compensation element, the calibration chamber can have a pressure gauge.

In one embodiment of the present disclosure, the container is designed as a flexible bag, into which bag a plurality of components of the calibration device are welded, wherein the components welded into a flexible bag are at least
the electrolyte liquid, the first electrode, and the second electrode,

and wherein the flexible bag has an upper end to which upper end the calibration chamber can be connected.

A great advantage of this embodiment is that by providing the electrolyte liquid in the flexible bag, the properties of the electrolyte liquid (for example, already purged with argon prior to being welded-in) can be controlled very easily. The bag may be provided to a user without the need for additional argon purging by the user.

The bag is designed, for example, as a single-use part (“disposable”) of an otherwise reusable calibration device which can be used, as a result of fresh provision of a flexible bag, for further calibration processes with the same calibration device.

The bag can then be installed, for example, in a holder of the calibration device provided for this purpose.

Ideally, the bag is sheathed (protection against, for example, the film tearing) and/or made from a thick-walled plastic. As a result, it can be placed on the floor, for example. Optionally, the bag is metal-coated.

In the case of an extremely dimensionally stable bag, a separate holder for the bag may not be necessary.

In one embodiment of the present disclosure, the flexible bag has at least one predetermined puncture point at the upper end.

The puncture point serves, for example, for connecting the calibration chamber to the gas-generation chamber in the event that the gas-generation chamber is part of the components welded into the bag.

Otherwise, the gas-generation chamber already connected to the calibration chamber can be introduced into the bag by means of the puncture point, for example, by inserting or screwing into the bag the gas-generation chamber taking the form of a tube.

In one embodiment of the present disclosure, the gas-generation chamber takes the form of one of the components welded into the flexible bag, wherein the liquid-impermeable and gas-permeable membrane is arranged at the upper end of the bag, wherein a sterile membrane is applied to the gas-permeable and liquid-impermeable membrane, which sterile membrane serves to protect the gas-permeable and liquid-impermeable membrane.

The sterile membrane is, for example, a welded-on film (similar to the aluminum foil of a milk carton) which serves for the sterile sealing of the bag and of the liquid-impermeable and gas-permeable membrane.

The present disclosure also relates to a flexible bag for providing components of a calibration device according to the present disclosure,

wherein the flexible bag forms the container of the calibration device, and at least welded into the flexible bag are:
the electrolyte liquid, the first electrode, and the second electrode,

and wherein the calibration chamber can be connected to an upper end of the bag.

In one embodiment, the material of the bag comprises a plastic film.

In one embodiment, a thickening agent is added to the electrolyte liquid.

The bag is especially designed as a so-called “disposable,” i.e., is especially usable for precisely one calibration.

With regard to the method for calibrating a sensor, the object is achieved by a method for calibrating a sensor, which sensor is designed to determine a proportion of a gas to be detected in a gas mixture, with a calibration device according to the present disclosure, comprising the following steps:
A) Introducing the sensor to be calibrated into the guide of the calibration chamber;
B) Applying an electric voltage to the electrodes, a gas mixture containing the gas to be detected being separated by means of electrolysis at the first electrode in the electrolyte liquid;
C) Transferring the gas mixture into the calibration chamber, during which transfer the gas mixture flows through the open closable opening into the calibration chamber and is thereby made available to the sensor to be calibrated;
D) Calibrating the sensor to be calibrated in the calibration chamber.

In one embodiment of the method, a multi-point calibration, especially, with at least two calibration points, is carried out in that steps B) to D) are carried out successively with respectively different applied electric voltages, wherein different concentrations of the gas to be detected in the gas mixture are in each case adjusted by the different applied electric voltages.

### BRIEF DESCRIPTION OF THE DRAWINGS

The present disclosure and further advantageous embodiments are explained in more detail below with reference to exemplary embodiments. The same parts are labeled with the same reference sign in all figures; for reasons of clarity or if it appears sensible for other reasons, reference signs used before are not repeated in the following figures.

The following are shown:

FIG. 1 shows a sectional view of an embodiment of the calibration device according to the present disclosure;

FIG. 2 shows a sectional view of a detail of another embodiment of the calibration device according to the present disclosure;

FIGS. 3a to 3c show details of another embodiment of the calibration device according to the present disclosure;

FIGS. 4a, 4b show various embodiments of a flexible bag with which components of the calibration device according to the present disclosure are provided, and

FIG. 5 shows a flow diagram of an embodiment of the method according to the present disclosure.

### DETAILED DESCRIPTION

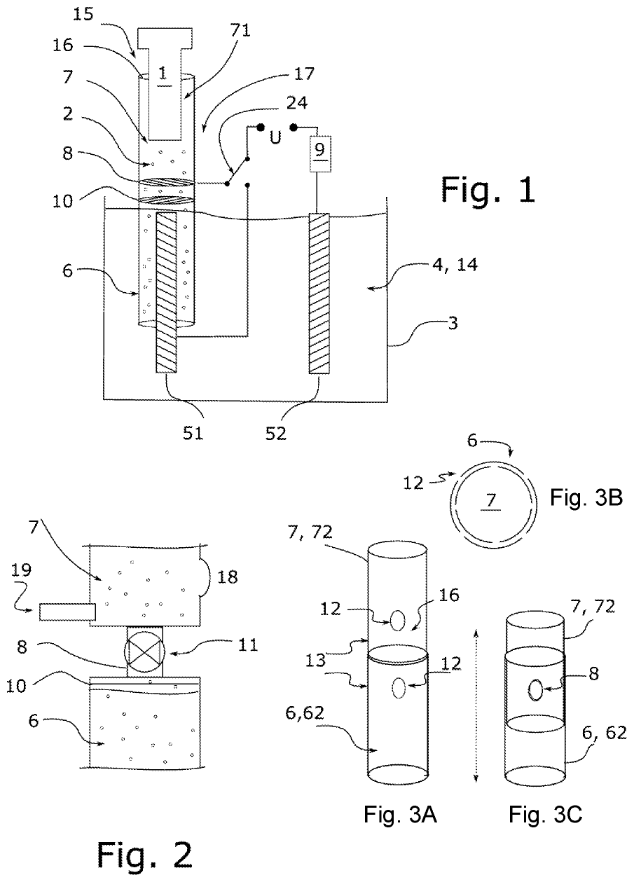

FIG. 1 shows the basic principle of the present disclosure in an embodiment of the calibration device according to the present disclosure.

The calibration device comprises a container 3 into which two electrodes 51, 52 are immersed in an electrolyte liquid 4 arranged therein. A catalyst 14 is optionally added to the electrolyte liquid 4. A first electrode 51 of the two electrodes 51, 52 is at least partially surrounded by a gas-generation chamber 6 which is here substantially cylindrical.

The gas-generation chamber 6 serves to collect the gas mixture generated at the first electrode 51 with a gas 2 to be detected.

In this exemplary embodiment, the first electrode 51 is the anode and the electrolyte liquid 4 is argon-purged urea. When a voltage U is applied, a gas mixture containing oxygen O2 is generated at the anode 51.

A variant for the production of inert gases at room temperature, investigated in tests by the applicant with the calibration device according to the present disclosure, is the electrolysis of urea solution at room temperature. Potentials around 1.5 V are needed to decompose an alkaline urea solution in a controlled manner into nitrogen, carbon dioxide. The following reaction equations can in principle be set up here:

Chemical Release:

CO(NH2)2+6OH−---->N2+5H2O+CO2+6e− Anode reaction:

6H2O+6e−----->3H2+6OH− Cathode reaction:

CO(NH2)2+H2O----->N2+3H2+CO2 Overall reaction:

Surprisingly, in the investigations by the applicant, in addition to the products in the reaction equations listed above, low 2 vol. % concentrations of oxygen O2 were measured every time at the anode 51. The proportion of oxygen O2 can be adjusted in a controlled manner by electrolysis and is therefore eminently suitable for generating gas mixtures which can be used as oxygen trace-sensor test gases. In this case, the gas 2 to be detected, oxygen O2, usually has a proportion of 0.001 to 21 percent by volume (or oxygen partial pressure from 0 to 50 hPa oxygen per nitrogen or nitrogen/carbon dioxides).

In the applicant's investigations, 1.5 V was applied as voltage U, i.e., the voltage U of a commercially available miniature battery. An alkaline urea solution having a concentration of 0.5 M urea and 5 M potassium hydroxide was used and the separated gas mixture at the anode and cathode was analyzed by means of gas chromatography. It was found for the collected gas mixtures and measurements in the gas chromatograph that, in addition to nitrogen N2 (96 vol. %) (see the anode reaction equation above), small proportions of hydrogen (2 vol. %) and small amounts of oxygen O2 (1.9 vol. %) also arise at the anode.

Here, the oxygen O2 as the gas 2 to be detected is presumably generated from hydroxides, for example, from an added catalyst 14, and/or is separated from the hydroxide of the anode 51 (for example, nickel hydroxide). For example, oxygen O2 may form in small amounts at the anode 51 due to oxyhydroxide formation or hydrogen peroxide formation and decomposition. The device (or the method described) used in the investigations of the applicant is therefore eminently suitable as a calibration device for a trace sensor, for example, for an oxygen or hydrogen trace sensor.

For this purpose, the gas mixture generated with the gas 2 to be detected is transferred from the gas-generation chamber 6 into a calibration chamber 7 connected thereto via a closable opening 8. A membrane 10, which is gas-permeable and liquid-impermeable, is arranged upstream (or even downstream) of the closable opening 8. The electrolyte liquid 4 thus remains in the gas-generation chamber 6, while the gas 2 to be detected or the gas mixture can flow into the calibration chamber 7.

For an on-site calibration, the calibration chamber 7 can then be placed as it were on a sensor 1 to be calibrated the sensor 1 via a guide 71 of the calibration chamber 7 for introducing the sensor.

The calibration chamber 7 has a closure 15 with which it can be sealed in a gas-tight manner. According to the present disclosure, calibration can thus be carried out in a calibration chamber 7 thus closed with a closure 15. However, this is not essential; for the case of an embodiment without a closure, calibration can be carried out, for example, in a calibration chamber 7 designed as a flow chamber. The closure 15 has, for example, a metal-coated polymer film 16 for sealing.

The applied voltage U can be controlled via a voltage converter 9. The proportion of oxygen O2 can be adjusted via the voltage U.

In the variant of the calibration device shown in FIG. 1, the closure 15, which mechanically couples together at least a closable opening 8 and a switch 24 of the power supply of the electrodes 51, 52 by means of a closure mechanism 17 in such a way that substantially simultaneously via a single operation, the closure 15 can be closed, the at least one closable opening 8 can be closed, and the power supply of the electrodes 51, 52 can be switched off.

In this way, the flow of the gas mixture from the gas-generation chamber 6 into the calibration chamber 7 (by closing the opening 8) and a possible outflow from the calibration chamber 7 into the environment (by closing the closure 15) can thus be terminated for the calibration, for example, at substantially the same time as the power interruption which terminates gas generation. It is thus possible to calibrate without any flow in a controlled manner (see Variant A in the tabular overview below).

| | A | B | C |
|---|---|---|---|
| | No-flow | Flow | Accumulation |
| | calibration | calibration | calibration |
| Closure | open −> closed | closed −> open | closed −> open |
| Opening | open −> closed | closed −> open | closed −> open |
| Power supply | on −> off | off −> on | on −> on |

Another variant involves a flow calibration (see Variant B).

In this case, the initially closed closure 15 and the initially closed opening 8 are simultaneously opened for the calibration, and the initially switched-off power supply is also switched on.

Another possibility is an accumulation calibration (see Variant C). In this case, the power supply is initially on and the opening 8 and the closure 15 are closed so that, as in the case of a bomb, the gas mixture can first collect in the gas-generation chamber 6. Only when enough gas mixture has collected in the gas-generation chamber 6 is the closed opening 8 and also the closure 15 opened for the calibration, and the gas mixture is thereby transferred into the calibration chamber 7. The sufficient gas accumulation can be checked, for example, by an additional pressure gauge (not shown) in the gas-generation chamber 6. The power supply can remain switched on in Variant C and therefore does not necessarily have to be mechanically coupled to the closure mechanism 17.

Independently of the variants mentioned above, the closable opening 8 can also have a valve 11 alternatively or in addition to the membrane 10; see FIG. 2. The valve 11 has the advantage that the gas flow from the gas-generation chamber 6 into the calibration chamber 7 can be controlled or regulated particularly easily.

In Variant C mentioned above, the valve 11 is preferably used to regulate the flow of the gas mixture collected in the gas-generation chamber 6 from the gas-generation chamber 6 into the calibration chamber 7, for example: initial stronger inflow, followed by weaker inflow. In the case in which a membrane 10 is also used in Variant C, it should withstand a water intrusion pressure of at least 3 bar, especially, at least 4 bar.

In the section shown in FIG. 2, a pressure gauge 19 is in addition also arranged in the calibration chamber 7. Furthermore, the calibration chamber 7 has a flexible pressure compensation element 18. In the simplest case, this can consist of an elastomer (“air balloon”). With the pressure gauge 19 or the pressure compensation element 18, a pressure rise caused by the inflow of the gas mixture from the gas-generation chamber 6 into the calibration chamber 7 can be measured or limited.

The gas-generation chamber 6 and the calibration chamber 7 (see FIGS. 3a to 3c) in one variant of the present disclosure take the form of two hollow bodies 62, 72 plugged into each other. They each have at least one recess 12 in their walls 13; see FIG. 3a. FIG. 3b shows a plan view of a cross-sectional area of the hollow bodies 62, 72 which in this embodiment are cylindrical and in which a multiplicity of recesses 12 arranged in the wall 13 are shown.

The opening 8 can thus be opened (FIG. 3c) or closed (FIG. 3a) by means of a relative movement, whereby the inflow of the gas mixture from the gas-generation chamber 6 into the calibration chamber 7 is controlled. The relative movement is a displacement along the common longitudinal axis of the cylindrical hollow bodies 62, 72, indicated by the dashed arrow between FIGS. 3a and 3c; however, a rotation about the longitudinal axis of the hollow bodies 62, 72 that are cylindrical in this embodiment and/or a displacement along this longitudinal axis is, for example, also possible.

The opening 8 and/or the above-mentioned closure 15 of the calibration chamber 7 can be sealed off by O-rings, a union sleeve with seal, ground glass joints, or the already mentioned film 16, etc. for a short calibration duration and seals off the sensor 1 and the cylindrical hollow body 72 of the calibration chamber 7, whereby the calibration chamber 7 and the gas flowing into it are shielded from the environment.

Depending on requirements, the calibration device can be made of materials such as stainless steel, plastic, glass, etc.

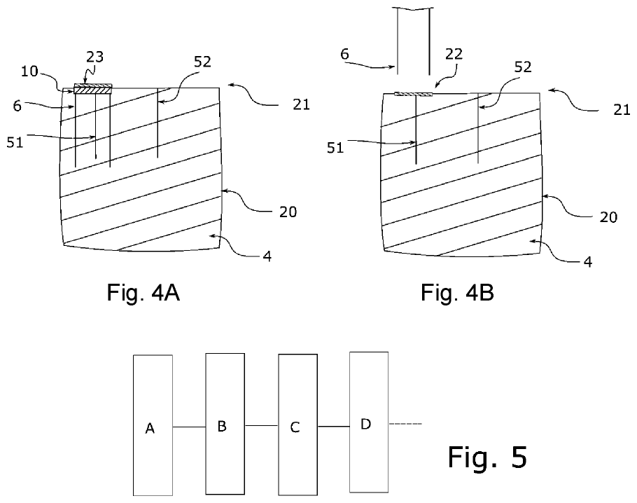

FIGS. 4a, 4b show a preferred variant of the present disclosure in which the container 3 with the electrolyte liquid 4 is provided by a flexible bag 20 into which at least the electrodes 51, 52 and the electrolyte liquid 4 are welded. The bag 20 is preferably designed as a disposable item, i.e., for one-time use in a calibration process. The bag 20 is made of a film which is preferably reinforced with a metal coating. The metal coating serves on the one hand to increase the mechanical stability of the bag 20 and on the other hand to increase its gas tightness.

In a first variant shown in FIG. 4a, the gas-generation chamber 6 (for example, as a thin plastic tube) is also welded into the bag 20. At an upper end 21, the gas-generation chamber 6 is closed off by the membrane 10, which in turn is protected by a sterile membrane 23. The bag 20 can simply be inserted into a holder and connected to the calibration chamber 7 at the upper end 21 of the bag so that the opening 8 is formed, the membrane 10 being arranged in the opening 8. In this variant, the holder and the calibration chamber 7 are thus reusable, but all of the components arranged in the bag 20 are designed to be disposable. Before the calibration device is put into operation, only the sterile membrane 23 which seals the bag 20 (similarly to a sterile membrane of a beverage carton) needs to be removed.

In a second variant shown in FIG. 4b, the gas-generation chamber 6 is not welded into the flexible bag 20 but can be introduced via a puncture point 22 into a predetermined position, for example, by a hollow cylindrical tube being rotatable into the bag 20 at the puncture point 22.

FIG. 5 shows a flowchart of an embodiment of the method according to the present disclosure.

In a first step A), the sensor 1 is introduced into the guide 71.

In a second step B), an electric voltage U is applied to the electrodes 51, 52. A gas mixture containing the gas 2 to be detected is separated by means of electrolysis at the first electrode 51 in the electrolyte liquid 4.

In a third step C), the gas mixture is transferred into the calibration chamber 7 in that it flows through the open closable opening 8 into the calibration chamber 7 and is thereby made available to the sensor 1 to be calibrated.

In a fourth step D), the sensor 1 to be calibrated is calibrated at a calibration point in the calibration chamber 7. The first calibration point (i.e., a specific concentration of the gas 2 to be detected) can be adjusted via the voltage U. The concentration of the gas 2 to be detected is known for a first adjusted first voltage U1 and/or can be determined with a reference sensor.

For a multi-point calibration, steps B) to D) are repeated for at least one further adjusted voltage U2, which leads to a second concentration of the gas 2 to be detected. This second concentration of the gas 2 to be detected forming a further calibration point is known again at the second voltage U2 and/or can be determined with a reference sensor.

1. A calibrating device for calibrating a sensor designed to determine a proportion of a gas in a gas mixture, the calibration device comprising:
a container having an electrolyte liquid and two electrodes immersed in the electrolyte liquid;
a gas-generation chamber arranged in the container such that the gas-generation chamber at least partially surrounds a first electrode of the two electrodes and a portion of the electrolyte liquid is in the gas-generation chamber; and
a calibration chamber having a guide into which the sensor to be calibrated can be at least partially introduced,
wherein the calibration chamber is connected via at least one closable opening to the gas-generation chamber,
wherein, by application of an electric voltage to the electrodes, the electrolyte liquid at the first electrode by means of electrolysis separates a gas mixture containing the gas to be detected, and
wherein the gas mixture flows from the gas-generation chamber through the open closable opening into the calibration chamber.

2. The calibration device according to claim 1,
wherein at the first electrode, the electrolyte liquid separates a gas mixture containing oxygen as the gas to be detected, the gas mixture having an oxygen proportion of 0.001 to 21 percent by volume.

3. The calibration device according to claim 2,
wherein the electrolyte liquid is an alkaline urea solution and the first electrode is an anode,
wherein the applied electric voltage has a value from a range of 0.2-1.9 volts, and
wherein the oxygen proportion is adjustable via the applied electric voltage.

4. The calibration device according to claim 3, further comprising:
a voltage converter for reducing the applied voltage to a value in a range between 0.2 and 1.4 volts.

5. The calibration device according to claim 1,
wherein the at least one closable opening includes a liquid-impermeable and gas-permeable membrane so that the gas mixture flows from the gas-generation chamber via the liquid-impermeable and gas-permeable membrane into the calibration chamber when the opening is open and the electrolyte liquid remains in the gas-generation chamber.

6. The calibration device according to claim 1,
wherein the at least one closable opening includes a valve.

7. The calibration device according to claim 1,
wherein the gas-generation chamber is designed as a first hollow body and the calibration chamber as a second hollow body.

8. The calibration device according to claim 7,
wherein a wall of the first hollow body and a wall of the second hollow body each have at least one recess, and
wherein the at least one closable opening between the gas-generation chamber and the calibration chamber is formed in that the at least one recess in the wall of the first hollow body can be brought into congruence with the at least one recess in the wall of the second hollow body by relative movement of the two hollow bodies.

9. The calibration device according to claim 1,
wherein the calibration chamber has a volume of less than 50 ml.

10. The calibration device according to claim 1,
wherein the electrolyte liquid includes a catalyst having metallic salts of the transition metals of the fourth period.

11. The calibration device according to claim 1,
wherein at least one of the electrodes comprises a metal selected from the group of the following or combinations thereof: platinum, titanium, iridium, nickel, and ruthenium.

12. The calibration device according to claim 1,
wherein the calibration chamber includes a closure by means of which the calibration chamber can be sealed off from an environment in a substantially gas-tight manner, and/or
wherein the at least one closable opening is sealed off in a substantially gas-tight manner when the opening is closed.

13. The calibration device according to claim 12,
wherein at least one closable opening incudes a metal-coated film for sealing off the calibration chamber from the gas-generation chamber in a substantially gas-tight manner, and/or
wherein the closure has a metal-coated film for sealing off the calibration chamber from the environment in a substantially gas-tight manner.

14. The calibration device according to claim 13,
wherein the closure and the at least one closable opening are mechanically coupled to each other via a closure mechanism such that the closure and the at least one closable opening can be operated simultaneously via a single operation of the closure mechanism.

15. The calibration device according to claim 14,
wherein a switch of a power supply of the electrodes is mechanically coupled to the closure mechanism so that the power supply of the electrodes can be operated via the single operation of the closure mechanism.

16. The calibration device according to claim 1, further comprising:
a pressure compensation element designed to compensate for a pressure rise in the calibration chamber.

17. The calibration device according to claim 1,
wherein the container is designed as a flexible bag into which bag a plurality of components of the calibration device are welded,
wherein the components welded into a flexible bag are at least the electrolyte liquid, the first electrode, and the second electrode, and
wherein the flexible bag has an upper end to which the calibration chamber can be connected.

18. The calibration device according to claim 17,
wherein the flexible bag has at least one predetermined puncture point at the upper end.

19. The calibration device according to claim 17,
wherein the gas-generation chamber is designed as one of the components welded into the flexible bag,
wherein the liquid-impermeable and gas-permeable membrane is arranged at the upper end of the bag, and
wherein a sterile membrane is applied to the gas-permeable and liquid-impermeable membrane, the sterile membrane serving to protect the gas-permeable and liquid-impermeable membrane.

20. A flexible bag for providing components of a calibration device, the flexible bag comprising:
an electrolyte liquid, a first electrode, and a second electrode,
wherein the flexible bag forms the container of a calibration device, and at least welded into the flexible bag are the electrolyte liquid, the first electrode, and the second electrode, and
wherein a calibration chamber can be connected to an upper end of the flexible bag.

21. A method for calibrating a sensor, wherein the sensor is designed to determine a proportion of a gas to be detected in a gas mixture, the method comprising:
providing a calibration device, including:
a container having an electrolyte liquid and two electrodes immersed in the electrolyte liquid;
a gas-generation chamber arranged in the container such that the gas-generation chamber at least partially surrounds a first electrode of the two electrodes and a portion of the electrolyte liquid is in the gas-generation chamber; and
a calibration chamber having a guide into which the sensor to be calibrated can be at least partially introduced,
wherein the calibration chamber is connected via at least one closable opening to the gas-generation chamber,
wherein, by application of an electric voltage to the electrodes, the electrolyte liquid at the first electrode by means of electrolysis separates a gas mixture containing the gas to be detected, and
wherein the gas mixture flows from the gas-generation chamber through the open closable opening into the calibration chamber;
introducing the sensor to be calibrated into the guide of the calibration chamber;
applying an electric voltage to the electrodes, a gas mixture containing the gas to be detected being separated by means of electrolysis at the first electrode in the electrolyte liquid;
transferring the gas mixture into the calibration chamber, during which transfer the gas mixture flows through the open at least one closable opening into the calibration chamber and is thereby made available to the sensor to be calibrated; and
calibrating the sensor to be calibrated in the calibration chamber.

22. The method according to claim 21,
wherein a multi-point calibration with at least two calibration points is carried out by repeating the applying of the electric voltage, the transferring of the gas mixture, and the calibrating of the sensor successively with respectively different applied electric voltages, and
wherein different concentrations of the gas to be detected in the gas mixture are in each case adjusted by the different applied electric voltages.
